(19) United States
(12) Patent Application Publication
(10) Pub. No.: US 2021/0322338 A1
(43) Pub. Date: Oct. 21, 2021

(54) COMPOSITIONS COMPRISING CBD FOR TREATING DERMATOLOGICAL CONDITIONS

(71) Applicant: UNV MEDICINE LTD., Ashkelon (IL)

(72) Inventors: Omer LAYANI, Ashkelon (IL); Ilana LAVON, Ashkelon (IL); Shaul MUKHTAR, Ashkelon (IL)

(21) Appl. No.: 17/275,264

(22) PCT Filed: Sep. 11, 2019
(86) PCT No.: PCT/IL2019/051017

§ 371 Date: Mar. 11, 2021

(30) Foreign Application Priority Data
Sep. 13, 2018 (IL) 261774

(51) Int. Cl. A61K 31/05; A61P 17/02; A61K 36/886; A61K 36/58; A61K 47/20; A61K 36/53; A61K 36/537; A61K 36/324; A61K 36/328; A61K 36/75; A61K 36/28; A61K 36/185; A61K 9/00

(52) U.S. Cl. CPC A61K 31/05; A61P 17/02; A61K 36/886; A61K 36/58; A61K 47/20; A61K 36/53; A61K 9/0014; A61K 36/324; A61K 36/328; A61K 36/75; A61K 36/28; A61K 36/185; A61K 36/537

(57) ABSTRACT

The present invention provides compositions and methods for the treatment of skin lesions. In particular, embodiments of the invention provide topical compositions useful in the treatment and alleviation of cutaneous symptoms associated with psoriasis, dermatitis and other dermatological conditions including ulcers or lesions, wherein the compositions contain CBD oil.

### FIELD OF THE INVENTION

The present invention relates to compositions comprising CBD useful in methods for the treatment and management of dermatological conditions, including psoriasis, dermatitis, skin ulcers and lesions.

### BACKGROUND OF THE INVENTION

Skin integrity is of importance for the protection and separation of body tissues from the surrounding environment. The loss of skin due to burns or trauma exposes the body to severe stress, impairing or even eliminating the many vital functions this organ performs. The skin contains two main layers of cells: a thin outer layer, the epidermis, and a thicker inner layer, the dermis. Full thickness skin tissue is comprised of keratinocytes lined on a basement membrane, produced by fibroblasts. Deeper layers of the skin include, in addition to fibroblasts, fat cells and multiple subsets of immune cells such as dendritic cells, lymphocytes and polymorphonuclear cells. The complex organization of normal skin is designed to support the numerous functions of this organ as both an immunologic and a physical barrier.

Many herbs and herbal preparations have been in use in traditional medicine, including preparations intended for the treatment of various skin disorders. However, the efficacy of these preparations in the treatment of dermatological conditions has not been sufficiently established. For example, while there are several Chinese herbal medicines intended for treating atopic eczema, there is no conclusive evidence that these treatments, taken by mouth or applied topically, reduce the severity of eczema in children or adults (Gu et al., Cochrane Database of Systematic Reviews 2013, Issue 9. Art. No.: CD008642). Specifically characterized formulations containing inter alfa herbal ingredients and various other substances and excipients have also been disclosed, for example US20090004301 relates to treating dyshidrosis and dry skin disorders, comprises topical administration of a composition comprising black walnut, wormwood, turmeric and garlic, antibacterial/anti-inflammatory herbs, and niacin, and oral administration of niacin.

The Cannabis plant has long been used for medicinal and recreational purposes alike. Subsequently, this plant has been characterized as containing many naturally occurring substances of great interest in the fields of science and medicine. The medical use of Cannabis is now legal in many countries under specific “medical Cannabis ” legislation. Medical Cannabis is used for treating and alleviating symptoms associated with a growing number of indications, including pain, anorexia, asthma, glaucoma, arthritis, spasms, anxiety, and substance withdrawal. Many other illnesses are emerging as potential Cannabis-responsive indications.

In recent years, researchers have also investigated the use of Cannabis for the treatment of certain dermatologic conditions, including pruritus and skin cancer; certain cannabinoids have also been suggested to manifest anti-inflammatory properties. Further studies are required to explore these potential uses. Conversely, the side effects of Cannabis use are relatively well documented, and include inter alfa Cannabis allergy manifesting as urticaria and pruritus, Cannabis arteritis presenting with necrosis and ulcers, and oral cancers from Cannabis smoke.

Certain topical formulations containing cannabinoids or other Cannabis-derived ingredients have been described. For example, US2016374958 discloses anti-bacterial and anti-fungal compositions in spray form, in cream form, in liquid form, and in powder form, comprising cannabinoids, specifically cannabidiol, cannabigerol, tetrahydrocannabinol, tetracannabidivarin, and/or cannabidivarin. The compositions are suggested to be used to treat toe nail fungus, MRSA infection, herpes virus infection, tinea pedis, burn wound infections, sun burns, diabetic infections, eczema, impetigo, dermatophytosis, psoriasis, itchy skin, atopic dermatitis, dandruff, and general topical infections.

WO2016133824 discloses cosmetics and topical formulations comprising inter alfa hemp oil containing cannabigerol, a non-psychoactive cannabinoid, and various other herbal oils and extracts that are characterized therein as being anti-oxidants, anti-microbial agents or anti-inflammatory agents. These topical and cosmetic formulations are suggested to have wound healing, skin firming, anti-bacterial, anti-dandruff, skin soothing, and UV ray protection properties.

Chelliah et al., (Pediatric Dermatology. 2018;1-4) described 3 cases of self-initiated topical cannabidiol use in patients with epidermolysis bullosa.

WO2018023164 describes a method for treating a skin disorder, comprising administering to a patient an effective amount of the pharmaceutical composition comprising a Cannabis extract and optionally one of more pharmaceutically acceptable carriers, wherein the Cannabis extract comprises at least 75% by weight of main cannabinoid.

There remains a medical need for safe and efficient formulations for the treatment and management of various dermatological conditions.

### SUMMARY OF THE INVENTION

The present invention in embodiments thereof relates to compositions and methods for the treatment of dermatological conditions, associated with skin lesions and ulcers, wherein the compositions include CBD oil. In particular, embodiments of the invention provide topical compositions useful in the treatment and alleviation of cutaneous symptoms associated with skin asthma, psoriasis, dermatitis and other dermatological conditions characterized by the appearance of ulcers or lesions.

The invention is based, in part, on the development of formulations, having unexpectedly advantageous properties in the treatment of skin lesions. In various embodiments, the compositions were found to exhibit marked and consistent efficacy and safety in the treatment of various skin lesions, and are in particular useful as topical medicaments. According to other embodiments, the compositions were found to exhibit improved absorption and tolerability, as described herein.

In particular, it was surprisingly discovered that compositions comprising oil extracts from cannabidiol (CBD)-rich (CBD-dominant) Cannabis strains were significantly more effective in alleviating skin lesions than compositions comprising tetrahydrocannabinol (THC)-dominant strains.

The compositions of the present invention comprise a unique combination of ingredients from Cannabis and other botanical sources. This unique combination provides advantageous properties over the use of each ingredient alone, which may be in various embodiments additive or greater than additive (synergistic), depending on the particular measured outcome.

According to embodiments of the invention, the composition comprises extracts obtained from Cannabis (Cannabis sativa L., e.g. Cannabis sativa and/or Cannabis indica strains approved for medical use).

According to one aspect, the composition of the present invention comprises CBD oil, e.g. from about 1 to about 30% by weight, Aloe vera extract, e.g. from about 10 to about 30% by weight, and Neem oil, e.g. from about 2 to about 10% by weight. According to some embodiments, the composition further comprises a dermatologically acceptable carrier.

According to some embodiments, the composition of the invention may include about 1-30% CBD oil by weight. According to some embodiments, the composition of the invention may include about 2-25% CBD oil by weight. According to some embodiments, the composition of the invention may include about 5-15% CBD oil by weight. According to some embodiments, the composition of the invention may include about 7.5-12.5% CBD oil by weight. According to some embodiments, the composition of the invention may include about 8-12% CBD oil by weight. According to some embodiments, the composition of the invention may include about 9-11% CBD oil by weight. In some exemplary embodiments, the composition of the invention may include about 10% CBD oil by weight.

According to a typical embodiment of the invention, the composition comprises CBD oil, namely a full-plant Cannabis oil extraction, typically from cannabidiol (CBD)-dominant strains. An exemplary CBD-dominant Cannabis strain comprises about 6-30 wt %, typically 10-20 wt % CBD, e.g. about 18 wt % CBD, and up to 5 wt %, typically 0.5-3 wt %, more typically 1-2 wt % each of tetrahydrocannabinol (THC) and cannabinol (CBN). For example, medical grade CBD oil as provided by the Israel Ministry of Health contains about 10-30 wt % CBD, in particular an average of 10 wt %, 15 wt %, 20 wt % or 24 wt % CBD, depending on the strain (wherein the initial extract may be diluted e.g. in olive oil or other pharmaceutically acceptable oils to obtain the determined concentration).

According to some embodiments, the CBD oil comprises from 30 to 85 wt %, from 35 to 80 wt %, from 40 to 75 wt %, from 45 to 70 wt %, from 50 to 65 wt %, from 45 to 80 wt %, or from 55 to 85 wt % of CBD.

In some embodiments, one or more cannabinoids may be added to the composition in purified form. For example, CBD-containing oil may be manufactured synthetically, by addition of CBD (and optionally other cannabinoids and/or Cannabis-derived compounds such as terpenes) to oil, which may subsequently be used in manufacturing the composition.

According to embodiments of the invention, the composition may further include Aloe vera (Aloe barbadensis miller). Typically, Aloe vera in the form of a juice (or in some instances gel) may be obtained (e.g. by mechanical extraction) from the inner layer of Aloe vera leaves. Thus, according to some embodiments, the composition includes Aloe vera extract. According to some embodiments of the invention, the composition may include 7.5-50%, typically 10-40%, more typically 15-30%, e.g. about 20% by weight Aloe vera gel or extract.

According to embodiments of the invention, the composition may include Neem oil, which is the vegetable oil pressed from the fruits and seeds of the neem (Azadirachta indica) tree. In some embodiments, the composition may include 0.5-15%, typically 1-10%, more typically 2-7.5%, e.g. about 5% by weight Neem oil.

According to some embodiments of the invention, the composition may further include at least one organosulfur compound. According to one embodiment, the organosulfur compound is selected from the group consisting of dimethyl sulfoxide (DMSO) and methylsulfonylmethane (MSM). According to a typical embodiment of the invention, the composition comprises 20-55%, typically 25-45%, e.g. about 40% by weight DMSO and/or MSM. Without being bound to any particular theory, it is estimated that use of the organosulfur compound in such concentrations may improve transdermal absorption of the active ingredients of the composition and consequently improve the therapeutic effect of the composition.

According to some embodiments, the compositions may further include one or more non-active excipients such as an emulsifier, e.g. selected from surfactants, emulsifying waxes and combinations thereof. According to one embodiment, the emulsifiers, surfactants and/or emulsifying waxes include, but not limited to: lecithin, glyceryl monostearate, glyceryl monooleate, cetyl alcohol, stearyl alcohol, cetostearyl alcohol, and the like or combinations thereof.

According to some embodiments, the composition may include about 0.5-15%, typically 1-10%, more typically 2-7.5%, e.g. about 5% by weight of emulsifier(s) such as emulsifying waxes and/or surfactant(s).

In some embodiments, the composition may further include Dittrichia viscosa oil extract. According to a typical embodiment the composition comprises about 0.5-15%, typically 1-10%, more typically 2-7.5%, e.g. about 5% by weight Dittrichia viscosa oil extract. According to one embodiment, the oil of the Dittrichia viscosa oil extract is a vegetable oil such as olive oil or any other dermatologically acceptable oil.

In some embodiments, the composition may further include Lavender oil extract. For example, the oil may any vegetable/ dermatologically acceptable oil, such as, olive oil. According to a typical embodiment the composition comprises about 0.5-15%, typically 1-10%, more typically 2-7.5%, e.g. about 5% by weight Lavender oil.

In some embodiments, the composition may further include Frankincense powder (resin powder). According to a typical embodiment, the composition may include about 0.5-15%, typically 1-10%, more typically 2-7.5%, e.g. about 5% by weight Frankincense powder.

In some embodiments, the composition may further include essential oils. In some embodiments, the essential oils may be a combination of one or more essential oils. In some embodiments, the essential oils may include Lavender, Clary sage and Myrrh. According to a typical embodiment the composition may include about 0.5-10%, typically 1-7.5%, more typically 2-5%, e.g. about 2% by weight of essential oil(s).

In some embodiments, the composition may further include additional whole plant extracts. In some embodiments, the whole plant extracts may be obtained from a combination of one or more plants. In some embodiments, the whole plant extracts may include extracts from Ruta chalepensis, Dittrichia viscosa, and Moringa oleifera. According to a typical embodiment, the composition may include about 0.5-10%, typically 1-7.5%, more typically 2-5%, e.g. about 3% by weight of whole plant extracts.

According to another aspect, the composition of the present invention is for use in treatment of a dermatological condition in a subject in need thereof. Thus, according to one embodiment, the present invention provides a composition comprising about 1 to about 30 wt % of CBD oil, from about 5 to about 50 wt % of Aloe vera extract, from about 0.5 to about 15% by weight of Neem oil, for use in treatment of a dermatological condition in a subject in need thereof. According to another embodiment, the present invention provides a composition comprising about 5-25% by weight of CBD oil, about 10-30% by weight of Aloe vera extract, and about 2-10% by weight of Neem oil, for use in treatment of a dermatological condition in a subject in need thereof. According to some embodiments, the composition comprises a dermatologically acceptable carrier.

According to some embodiments, the composition may include about 8-12% by weight or about 10% by weight of CBD oil. According to some embodiments, the CBD oil may include about 30 to 85 wt % of CBD. According to other embodiments, the CBD oil may include about 55 to 85 wt % of CBD. According to some embodiments, the CBD oil may be obtained from plant extract, or may be prepared synthetically.

In some embodiments, the composition may include about 20% by weight of Aloe vera gel. In some embodiments, the composition may include about 5% Neem oil. In some embodiments, the composition may further include at least one organosulfur compound selected from the group consisting of dimethyl sulfoxide (DMSO) and methylsulfonylmethane (MSM). According to some embodiments, the at least one organosulfur compound comprise about 20-55% by weight of the composition. In some embodiments, the least one organosulfur compound comprises about 40% by weight of the composition. In some embodiments, the composition may further include about 2-10% by weight of surfactants. In some embodiments, the surfactants may be about 5% by weight of the composition. In some embodiments, the composition may further include about 2-10% by weight of Dittrichia viscosa in oil and/or Lavender in oil. In some embodiments, the Dittrichia viscosa in oil and/or Lavender in oil may each be about 5% by weight of the composition. In some embodiments, the composition may further include about 2-10% by weight of Frankincense powder. In some embodiments, the Frankincense powder may be about 5% by weight of the composition. In some embodiments, the composition may further include about 0.5-5% by weight of essential oils, selected from the group consisting of: Lavender, Clary sage and Myrrh. In some embodiments, the essential oils may be about 2% by weight of the composition. In some embodiments, the composition may further include about 0.5-5% by weight of whole plant extracts, said plants are selected from the group consisting of: Ruta chalepensis, Dittrichia viscosa, and Moringa oleifera. In some embodiments, the whole plant extracts may be about 3% by weight of the composition.

In some embodiments, the composition is a dermatological composition. In some embodiments, the composition is a pharmaceutical composition comprising one or more pharmaceutically acceptable excipients.

In some embodiments, the composition or the pharmaceutical composition is formulated for topical administration. In some embodiments, the formulation is in the form of an ointment, a gel, a lotion, skin patch or cream. According to one embodiment, the pharmaceutical composition is a dermatological pharmaceutical composition.

In some embodiments, the dermatological condition may be selected from: atopic dermatitis, skin cancer lesions, psoriasis, herpes zoster, benign hyperproliferative disorders, typically superficial wounds, shallow ulcers, or combinations thereof.

According to some embodiments, there is provided a method for treating a dermatological condition in a subject in need thereof, the method comprising administering to the subject a composition comprising about 5-25% by weight of CBD oil, about 10-30% by weight of Aloe vera extract, and about 2-10% by weight of Neem Oil.

Other objects, features and advantages of the present invention will become clear from the following description and drawings.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 shows pictures of a skin region of a subject afflicted with atopic dermatitis, prior to treatment (Day 0—FIG. 1A) and following topical administration of the composition disclosed herein (Day 8—FIG. 1B).

FIG. 2 shows pictures of a skin region of a cancer patient having skin lesions, prior to treatment (Day 0—FIG. 2A) and following topical administration of Formulation #6 on Day 7 (FIG. 2B) and Day 21 (FIG. 2C).

FIG. 3 shows pictures of a facial skin region of a cancer patient having skin lesions, prior to treatment (Day 0—FIG. 3A) and following topical administration of Formulation #6 on Day 7 (FIG. 3B) and Day 21 (FIG. 3C).

FIG. 4 shows pictures of a skin region of an ankle of a subject afflicted with psoriasis, prior to treatment (Day 0—FIG. 4A) and following topical administration of Formulation #6 (Day 2—FIG. 4B).

FIG. 5 shows pictures of a skin region of a subject afflicted with Shingles, prior to treatment (Day 0—FIG. 5A) and following topical administration of Formulation #6 (Day 4—FIG. 5B);

FIG. 6 shows pictures of a skin region of a subject afflicted with Shingles, prior to treatment (Day 0—FIG. 6A) and following topical administration Formulation #6 on Day 3 (FIG. 6B) and Day 5 (FIG. 6C).

### DETAILED DESCRIPTION OF THE INVENTION

The present invention provides compositions and methods for treating dermatological conditions, in particular, skin conditions associated with ulcers or lesions, by utilizing a composition which include CBD oil and one or more additional ingredients. The compositions and methods disclosed herein are particularly efficient, useful and safe with diminished side effects, as demonstrated herein. Topical administration of the compositions results in an efficient and rapid improvement of the treated condition, ulcer or lesion.

According to one aspect, the present invention provides a composition comprising from about 1 to about 30 wt % of CBD oil, from about 5 to about 50 wt % of Aloe vera extract, from about 0.5 to about 15% by weight of Neem oil. According to some embodiments, the composition further comprises a dermatologically acceptable carrier.

### Definitions

To facilitate an understanding of the present invention, a number of terms and phrases are defined below. It is to be understood that these terms and phrases are for the purpose of description and not of limitation, such that the terminology or phraseology of the present specification is to be interpreted by the skilled artisan in light of the teachings and guidance presented herein, in combination with the knowledge of one of ordinary skill in the art.

The term “cannabinoid” as used herein generally refers to one of a class of diverse chemical compounds that act on a cannabinoid receptor in cells that repress neurotransmitter release in the brain. The term “cannabinoid” as used herein further refers a chemical compounds that acts on cannabinoid receptors or has a structure similar the stature of a compound acting on cannabinoid receptor in cells. Ligands for these receptor proteins include the endocannabinoids (produced naturally in the body by humans and animals), the phytocannabinoids (found in Cannabis and some other plants), and synthetic cannabinoids (manufactured artificially).

The term “extract” as used herein refers a product prepared by extraction by physical means (e.g. by comminuting, pressing, heating, pulsed electric field assisted treatments, shear treatments and pressure wave treatments), by chemical means (e.g. by treatment with an acid, a base, a solvent) and/or by biochemical means (e.g. by treatment with hydrolytic enzymes, microorganisms). The term refers to a liquid substance obtained through extraction from a given substance, or to a concentrate or essence which is free of, or substantially free of solvent. The term extract may be a single extract obtained from a particular extraction step or series of extraction steps or the extract also may be a combination of extracts obtained from separate extraction steps or separate feedstocks. Such combined extracts are thus also encompassed by the term “extract”. Any method of extraction and any solvent is encompassed. The extract may be obtained from any part of the plant e.g. from leaves, flowers, stems, roots, fruits and seeds. The extract may be aqueous or oily. Commonly used solvents are water, ethanol, ethyl acetate, CO₂, methanol, acetone, and acetic acid. According to one embodiment, the solvent is ethanol. According to another embodiment, the extraction is by CO₂. In particular, the term “extract” refers to a liquid or semi-solid or resinous substance obtained through extraction from plants defined in the present application, i.e. extracts obtained from Cannabis plant, Dittrichia viscosa, Lavender, Clary sage, Myrrh, Ruta chalepensis, Moringa oleifera and Aloe vera. In some embodiments, the term refers to a mixture of liquid or semi-solid, resinous substances obtained through extraction from two or more different plans. In some embodiments, the term refers also to a compound purified from the extract. According to some embodiments, the term “extract” has the meaning of a mixture or combination of two or more extracts. The extract also may be formulated into a pharmaceutical composition or food product, as described further below.

The term “Cannabis extract” as used herein refers to one or more plant extracts from the Cannabis plant. A Cannabis extract contains, in addition to one or more cannabinoids, one or more non-cannabinoid components which are co-extracted with the cannabinoids from the plant material. Their respective ranges in weight will vary according to the starting plant material and the extraction methodology used. Cannabinoid-containing plant extracts may be obtained by various means of extraction of Cannabis plant material. Such means include but are not limited to: supercritical or subcritical extraction with CO2, extraction with hot or cold gas and extraction with solvents. In some embodiments, the term refers to a mixture of liquid or semi-solid, resinous substances obtained through extraction from two or more different Cannabis species. In some embodiments, the term refers also to a compound purified from the extract. The term “Cannabis plant” as used herein, refers to plants of the genus Cannabis, including but not limited to Cannabis sativa, Cannabis indica, and Cannabis ruderalis. The term “natural cannabinoid” as used herein generally refers to a cannabinoid which can be found in, isolated from and/or extracted from a natural resource, such as plants. “Synthetic cannabinoids” are a class of chemicals that are different from the cannabinoids found e.g. in Cannabis but which also bind to cannabinoid receptors.

In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV) and cannabigerol monomethyl ether (CBGM), salts thereof, derivatives thereof and mixtures of cannabinoids. Each possibility represents a separate embodiment of the invention.

The terms “cannabidiol” and “CBD” are interchangeably used herein and refer to a non-psychotropic cannabinoid having structure as described in Formula I below, salt or derivatives thereof, such as Δ4-cannabidiol, Δ5-cannabidiol, Δ6-cannabidiol, Δ1,7-cannabidiol, Δ1-cannabidiol Δ2-cannabidiol Δ3-cannabidiol.

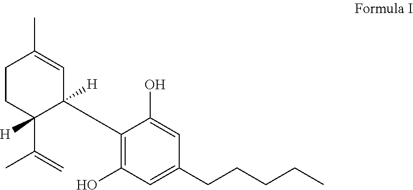 [SMILES: C=C(C)[C@@H]1CCC(C)=C[C@H]1c1c(O)cc(CCCCC)cc1O]

As used herein the term “CBD oil” is directed to a lipophilic Cannabis plant extract which comprises CBD and optionally one or more additional cannabinoids, such as, for example, but not limited to: THC, CBN, and the like. According to some embodiments, CBD is the main component in the CBD oil. The term CBD oil further encompasses oil formulations including pure cannabinoids (for example, synthetic forms), including CBD and optionally one or more additional cannabinoids. According to some embodiments, the term “CBD oil” encompasses also crude extract from Cannabis plants, in particular CBD rich strain, a fraction of the Cannabis extract comprising high concentration of CBD, or purified CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some such embodiments, the CBD:THC weight ratio in the CBD oil is at least 10:1, at least 15:1 or at least 20:1. According to some embodiments, the CBD:THC weight ratio is from 20:1 to 1:1 or from 20:1 to 1:1.

The term “essential oils” as used herein refers to a concentrated hydrophobic liquid containing volatile aroma compounds from a plant. Essential oils are also known as volatile oils, ethereal oils, aetherolea, or the oil of the plant from which they were extracted. As used herein, the term “fat” refers to saturated, mono-unsaturated and poly-unsaturated fatty acid.

Fatty acids are usually present in the form of esters (e.g. mono-/di-/triglycerides). As used herein the term “oil” is used as a generic term for lipids, fats, or any mixture thereof.

The term “pharmaceutical composition” as used herein has its conventional meaning and refers to a composition which is pharmaceutically acceptable. The term “pharmaceutically acceptable” as used herein has its conventional meaning and refers to compounds, material, compositions and/or dosage forms, which are, within the scope of sound medical judgment suitable for contact with the tissues of mammals, especially humans, without excessive toxicity, irritation, allergic response and other problem complications commensurate with a reasonable benefit/risk ratio. Typically, pharmaceutical composition comprises a pharmaceutically acceptable excipient. The term “excipient”, as used herein, has its conventional meaning and refers to a pharmaceutically acceptable ingredient, which is commonly used in the pharmaceutical technology for preparing a suitable dosage formulation, such as, topical formulation. The term “pharmaceutically acceptable carrier” or “pharmaceutically acceptable excipient” include any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The term “carrier” refers to a diluent, adjuvant, excipient, or vehicle with which the therapeutic is administered. Such carriers can be sterile liquids, such as water or oils, including those of petroleum, animal, vegetable or synthetic origin, such as peanut oil, soybean oil, mineral oil, sesame oil and the like, polyethylene glycols, glycerine, propylene glycol or other synthetic solvents. The carrier of the present invention is compatible with the skin, the mucous membranes and/or the skin appendages. According to some embodiments, the carrier is a pharmaceutically acceptable carrier. According to other embodiment, the carrier is a cosmeceutical or dermatologically acceptable carrier. The term “cosmetic composition” is intended to mean a substance or a preparation intended to be brought into contact with the various superficial parts of the body, in particular the epidermis, the body-hair and head- hair systems, the nails, the lips and the oral mucous membranes. The term “dermatological composition” is intended to mean a composition such as a pharmaceutical composition applied to the skin. The term “dermatologically acceptable carrier” is intended to mean those carriers which can safely be used on the skin in the topical treatment methods of this invention, i.e., carriers which do not provoke a severe reaction and which are not toxic when contacted with the skin.

The term “topical administration” or “topical application” refers to directly layering or spreading upon epidermal tissue, especially outer skin or membrane, including the skin or membrane of the oral, rectal or vaginal cavities.

The term “dosage form” denotes any form of the formulation that contains an amount of a cannabinoid or of a mixture of cannabinoids sufficient to achieve at least a partial therapeutic effect when appropriately administered. In some embodiments, the dosage form is of topical formulation. According to some embodiments, the topical formulation includes, but not limited to: cream, ointment, gel, solution, skin patch, lotion and balm.

As used herein when referring to the invention, the term “organosulfur compound(s)” refers to any compound containing carbon, hydrogen, and sulfur, where the sulfur is directly bonded to at least one carbon, wherein the compound is suitable for administration to human.

The term “emulsifier” as used herein are amphiphilic molecules being surface active agents and that are capable of reducing and/or eliminating the surface tension and the interfacial tension in a two-phase system and therefore stabilizing emulsion. The term emulsifier encompasses phospholipids, emulsifying wax, surfactants and combination thereof.

As used herein, the phrase “emulsifying agent” refers to a substance that is soluble in both oil and water and that enables oil(s) to be uniformly dispersed in water as an emulsion. For example, an emulsifying agent is commonly an emulsifying wax.

The term “emulsifying wax” denotes a solid nonionic emulsifier known in the art that are prepared as a mixture of fatty alcohols having from about 12 to about 24 carbon atoms, preferably predominantly lipophilic fatty alcohols having from about 14 to about 20 carbon atoms. Typically emulsifying wax comprise (prepared from) cetearyl alcohol and a polyoxyethylene derivative of a fatty acid ester of sorbitan. Non limiting examples of emulsifying wax are cetostearyl alcohol and polyoxyethylene sorbitan monostearates. According to some embodiments, the emulsifying wax is emulsifying wax NF. According to some embodiments, the emulsifying wax is emulsifying wax BP.

The term “surfactant” used in this invention has the meaning commonly understood in the art. Surfactants are used to both help facilitate the formation of the emulsion and improve its stability. Anionic, cationic, amphoteric, zwitterionic, nonionic surfactants and mixtures hereof may all be used in this invention.

The terms “treat,” “treating,” or “treatment” as used herein, include reducing, alleviating, ablating, ameliorating, relieving, or lessening a symptom associated with either a chronic or acute therapeutic scenario treatable with the composition of the invention. In some embodiments, the term includes, but are not limited to, alleviation or amelioration of one or more symptoms or parameters associated with a skin condition, such as improvement in parameters as assessed by various rating scales, tests, appearance and/or indices.

The terms “skin condition” and “dermatological condition” are used herein interchangeably and refer to any condition relating to diseased and/or damaged skin. Non-limiting examples of dermatological conditions include atopic dermatitis, allergic contact dermatitis, irritant contact dermatitis, radiation dermatitis, seborrheic dermatitis, psoriasis, sunburn, diabetic ulcers, pressure ulcers, and stasis ulcers. Dermatological conditions can also arise from irritation and/or pain following laser or chemical resurfacing, dermabrasion therapy, cuts, burns, and abrasions. According to some embodiments, the dermatological conditions are characterized by the appearance of ulcers or lesions, caused by various conditions and factors, including, but not limited to: atopic dermatitis, skin cancer lesions, psoriasis, herpes zoster, benign hyperproliferative disorders, typically superficial wounds or shallow ulcers, and the like.

The terms “% by weight” and “wt %” are used herein interchangeably and have the meaning commonly used in the field of pharmaceutical preparations.

The term “about” as used herein refers to any value which lies within a range of ±10% of original value. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

In some embodiments, the cannabinoid is a natural cannabinoid. In some embodiments, the cannabinoid is a natural cannabinoid found in a Cannabis plant. In some embodiments, the cannabinoid is a synthetic cannabinoid. In some embodiments, the cannabinoid is a mixture of natural cannabinoids. In some embodiments, the cannabinoid is a mixture of synthetic cannabinoids. In some embodiments, the cannabinoid is a mixture of natural and synthetic cannabinoids.

In some embodiments, the pharmacologically active cannabinoid may be selected from the group consisting of cannabidiol (CBD), tetrahydrocannabinol, Δ9-tetrahydrocannabinol (THC), Δ8-tetrahydrocannabinol, standardized marijuana extracts, Δ8-tetrahydrocannabinol-DMH, Δ9-tetrahydrocannabinol propyl analogue (THCV), 11-hydroxy-tetrahydrocannabinol, 11-nor-9-carboxy-tetrahydrocannabinol, 5′-azido-.Δ8-tetrahydrocannabinol, AMG-1 (CAS Number 205746-46-9), AMG-3 (CAS Number 205746-46-9), AM-411 (CAS Number 212835-02-4), (−)-11-hydroxy-7′-isothiocyanato-48-THC (AM-708), (−)-11-hydroxy-7′-azido-Δ8-THC (AM-836), AM-855 (CAS Number 249888-50-4), AM-919 (CAS Number 164228-46-0), AM926, AM-938 (CAS Number 303113-08-8), cannabidiol (CBD), cannabidiol propyl analogue (CBDV), cannabinol (CBN), cannabichromene, cannabichromene propyl analogue, cannabigerol, CP 47,497 (CAS Number (1S,3R): 114753-51-4), CP 55,940 (CAS Number 83002-04-4), CP 55,244 (CAS Number 79678-32-3), CT-3 (ajulemic acid), dimethylheptyl HHC, HU-210 (1, 1-Dimethylheptyl -11-hydroxy-tetrahydrocannabinol), HU-211 (CAS Number 112924-45-5), HU-308 (CAS Number 1220887-84-2), WIN 55212-2 (CAS Number 131543-22-1), desacetyl-L-nantradol, dexanabinol, JWH-051 (Formula C25H3802), levonantradol, L-759633 (Formula C26H40O2), nabilone, O-1184, and mixtures thereof. Each possibility is a separate embodiment.

According to some embodiments, the composition of the invention may include from about 1 to about 30% CBD oil by weight. According to some embodiments, the composition of the invention may include about 2-25% CBD oil by weight. According to other embodiments, the composition of the invention comprises from about 3 to about 20% of CBD oil by weight. According to some embodiments, the composition of the invention may include about 5-15% CBD oil by weight. According to one embodiment, the composition of the invention comprises from about 5 to about 25% of CBD oil by weight. According to some embodiments, the composition of the invention may include about 7.5-12.5% CBD oil by weight. According to some embodiments, the composition of the invention may include about 8-12% CBD oil by weight. According to some embodiments, the composition of the invention may include about 8 to about 15% of CBD oil by weight. According to some embodiments, the composition of the invention may include about 9-11% of CBD oil by weight. In some exemplary embodiments, the composition of the invention may include about 10% CBD oil by weight.

In some embodiments, the CBD oil may include from about 2% to about 90% CBD by weight. In some embodiments, the CBD oil may include from about 2% to about 75% CBD by weight. In some embodiments, the CBD oil may include from about 2% to about 60% CBD, by weight. In some embodiments, the CBD oil may include from about 2%-to about 50% CBD. In some embodiments, the CBD oil may include from about 2% to about 40% CBD by weight. In some embodiments, the CBD oil may include from about 2% to about 30% CBD by weight. In some embodiments, the CBD oil may include from about 2% to about 20% CBD by weight. In some embodiments, the CBD oil may include from about 2% to about 15% CBD by weight. In some embodiments, the CBD oil may include from about 5% to about 75% CBD by weight. In some embodiments, the CBD oil may include from about 5% to about 50% CBD by weight. In some embodiments, the CBD oil may include from about 5% to about 30% CBD by weight. In some embodiments, the CBD oil may include from about 5% to about 20% CBD by weight. In some embodiments, the CBD oil may include from about 7.5% to about 25% CBD by weight. In some embodiments, the CBD oil may include from about 7.5% to about 20% CBD by weight. In some embodiments, the CBD oil may include from about 7.5% to about 15% CBD by weight. In some embodiments, the CBD oil may include from about 10% to about 20% CBD by weight. In some embodiments, the CBD oil may include from about 20% to about 40% CBD by weight. In some embodiments, the CBD oil may include from about 40% to about 60% CBD by weight. In some embodiments, the CBD oil may include from about 50% to about 90% CBD by weight. In some embodiments, the CBD oil may include from about 60% to about 80% CBD by weight. In some embodiments, the CBD oil may include from about 30% to about 60% CBD by weight. According to some embodiments, the CBD oil comprises from 55 to 80 wt % of CBD. According to some embodiments, the CBD oil comprises from 40 to 80 wt % of CBD. According to some embodiments, the CBD oil comprises less than 5 wt %, less than 3 wt %, less than 2 wt %, 1 wt %, less than 0.5 wt %, less than 0.2 wt % or less than 0.1 wt % of THC. According to some such embodiments, the CBD:THC weight ratio in the CBD oil is at least 10:1, at least 15:1 or at least 20:1. According to some embodiments, the CBD:THC weight ratio is from 20:1 to 1:1 or from 20:1 to 1:1.

In some embodiments of the pharmaceutical composition, the natural cannabinoid is derived or isolated from an extract of a Cannabis plant. In some embodiments of the composition, the natural terpene is derived or isolated from an extract of a Cannabis plant.

In some embodiments, the pharmaceutical composition or the CBD oil may further include one or more cannabinoids in addition to CBD. In some embodiments, the one or more cannabinoids are selected from the group consisting of Cannabidiolic acid (CBDA), Tetrahydrocannabinol (THC), Tetrahydrocannabinolic acid (THCA), Cannabigerol (CBG), Cannabichromene (CBC), Cannabinol (CBN), Cannabielsoin (CBE), iso-Tetrahydrocannabimol (iso-THC), Cannabicyclol (CBL), Cannabicitran (CBT), Cannabivarin (CBV), Tetrahydrocannabivarin (THCV), Cannabidivarin (CBDV), Cannabichromevarin (CBCV), Cannabigerovarin (CBGV) and Cannabigerol Monomethyl Ether (CBGM), salts thereof, and derivatives thereof.

In some embodiments, the composition or the CBD oil comprises about 30% to about 80% by weight of a cannabinoid or a mixture of cannabinoids. In certain embodiments, the composition or the CBD oil comprises about 40% to about 70% by weight of a cannabinoid or a mixture of cannabinoids. In certain embodiments, the cannabinoid is selected from the group consisting of cannabidiol (CBD), cannabidiolic acid (CBDA), tetrahydrocannabinol (THC), tetrahydrocannabinolic acid (THCA), cannabigerol (CBG), cannabichromene (CBC), cannabinol (CBN), cannabielsoin (CBE), iso-tetrahydrocannabimol (iso-THC), cannabicyclol (CBL), cannabicitran (CBT), cannabivarin (CBV), tetrahydrocannabivarin (THCV), cannabidivarin (CBDV), cannabichromevarin (CBCV), cannabigerovarin (CBGV), cannabigerol monomethyl ether (CBGM), salts thereof, derivatives thereof and mixtures of cannabinoids.

In some embodiments, the amounts of the active ingredients may be determined by the skilled artisan accordingly to meet the desired concentration in the final product. Thus, for example, the concentration of CBD (e.g. in the oil used as a starting material) may be determined such that the final composition of CBD in the composition may contain about 0.5 to 25, 1 to 23%, 2 to 22%, 3 to 21%, 4 to 20, 5 to 18, 7 to 15,%, 0.5 to 12, 0.5-10%, 0.5-8%, 1-5%, 1-3%, e.g. 1.8% by weight CBD.

According to some embodiments, statement of CBD oil content in the composition may be replaced by statement of CBD content in the composition. Thus, according to some embodiments, the composition of the present invention comprises from about 0.5 to about 25 wt % of CBD, from about 5 to about 50 wt % of Aloe vera extract, from about 0.5 to about 15% by weight of Neem oil. According to some embodiments, the composition further comprises a dermatologically acceptable carrier. According to other embodiment, the composition comprises from 1 to 22, from 2 to 21 or from 4 to 15 wt % of CBD.

In some embodiments, the composition comprises at least about 20 mg of a cannabinoid or a mixture of cannabinoids, per lgr of composition. In some embodiments, the composition comprises at least about 50 mg of a cannabinoid or a mixture of cannabinoids, per lgr of composition. In some embodiments, the dosage form comprises about 20 mg to about 800 mg of a cannabinoid or a mixture of cannabinoids, per 1 gr of composition. In some embodiments, the composition comprises at least about 20-500 mg or about 20-150 mg of a cannabinoid or a mixture of cannabinoids, per lgr of composition. In certain embodiments, the dosage form comprises about 20 mg, 50 mg, 100 mg, about 200 mg, about 300 mg, about 400 mg, about 500 mg or about 600 mg of a cannabinoid or a mixture of cannabinoids, per 1 gr of composition.

In some embodiments, one or more cannabinoids may be added to the composition in purified form. For example, CBD-containing oil may be manufactured synthetically, by addition of CBD (and optionally other cannabinoids and/or Cannabis-derived compounds such as terpenes) to oil, which may subsequently be used in manufacturing the composition.

According to embodiments of the invention, the composition comprises Aloe vera (Aloe barbadensis miller). Typically, Aloe vera in the form of a juice or gel may be obtained (e.g. by mechanical extraction) from the inner layer of Aloe vera leaves. Thus, the term “Aloe vera extract” refers to juice and/or gel obtained from the inner layer of Aloe vera leaves. According to some embodiments of the invention, the composition may include about 7.5-50% by weight of Aloe vera extract. In some embodiments, the composition may include about 10-40% by weight of Aloe vera extract. In some embodiments, the composition may include about 12.5-35% by weight of Aloe vera extract. In some embodiments, the composition may include about 15-30% by weight of Aloe vera extract. In some embodiments, the composition may include about 15-25% by weight of Aloe vera extract. In some embodiments, the composition may include about 20% by weight of Aloe vera extract.

According to some embodiments, the composition of the invention includes Neem oil, which is the vegetable oil obtained (e.g. pressed) from the fruits and seeds of the neem (Azadirachta indica) tree. In some embodiments, the composition comprises about 0.5-15% by weight of Neem oil. In some embodiments, the composition comprises about 1-10% by weight of Neem oil. In some embodiments, the composition comprises about 2-7.5% by weight of Neem oil. In some embodiments, the composition comprises about 5% by weight Neem oil.

According to some embodiments, the composition of the invention further includes at least one organosulfur compound. According to one embodiment, the organosulfur compound is selected from the group consisting of dimethyl sulfoxide (DMSO) and methylsulfonylmethane (MSM). Both dimethyl sulfoxide (DMSO, an organic form of sulphur commercially prepared e.g. from lignin) and its oxidized form, methylsulfonylmethane (MSM, occurring e.g. in green plants fruits and vegetables) have been in use as solvents, as pharmaceutical carriers or excipients, and as dietary supplements suggested to have certain medical benefits. In some embodiments, the composition of the invention comprises from 15 to 65 wt %, or any subranges thereof, of at least one organosulfur compound. In some embodiments, the composition of the invention comprises from about 30% to about 50% by weight of at least one organosulfur compound. In some embodiments, the composition of the invention of may include about 25%-45% by weight of at least one organosulfur compound. In some embodiments, the composition of the invention of may include about 20%-40% or 20-60% by weight of at least one organosulfur compound. In some embodiments, the composition of the invention may include about 35%-45% by weight of at least one organosulfur compound. In some embodiments, the composition of the invention may include about 40% by weight of at least one organosulfur compound. In some embodiments, the composition of the invention of may include over about 20% by weight of at least one organosulfur compound. In some embodiments, the composition of the invention of may include about 15%-65% by weight, or any subranges thereof, of DMSO and/or MSM. In some embodiments, the composition of the invention may include about 20%-50% by weight of DMSO and/or MSM. In some embodiments, the composition of the invention may include about 25%-45% by weight of DMSO and/or MSM. In some embodiments, the composition of the invention may include about 20%-40% by weight of DMSO and/or MSM. In some embodiments, the composition of the invention may include about 35%-45% by weight of DMSO and/or MSM In some embodiments, the composition of the invention may include over about 20% by weight of DMSO and/or MSM. In some embodiments, the composition of the invention may include about 40% by weight of DMSO and/or MSM. According to one embodiment, the composition comprises from about 20 to about 55 wt % of DMSO. According to another embodiment, the composition comprises from about 30 to about 55 wt % of DMSO. According to a further embodiment, the composition comprises from about 20 to about 55 wt % of MSM. According to yet another embodiment, the composition comprises from about 30 to about 55 wt % of DMSO. According to any one of the above embodiments, the concentration of the at least one organosulfur compound does not exceed concentration allowed by the relevant regulatory authorities such as FDA or EMA.

According to some embodiments, the composition of the present invention comprises from about 1 to about 30 wt % of CBD oil, from about 5 to about 50 wt % of Aloe vera extract, from about 0.5 to about 15% by weight of Neem oil, from 20 to about 55% by weight of at least one organosulfur compound. According to another embodiment, the composition comprises from about 5 to about 25% by weight of CBD oil, from about 10 to about 30% by weight of Aloe vera extract, from about 2 to about 10% by weight of Neem oil, from about 20 to about 55% by weight of at least one organosulfur compound. According to some embodiments, the composition further comprises a dermatologically or a pharmaceutically acceptable carrier.

According to some embodiments, the composition of the invention may further include one or more emulsifier such as surfactant and/or emulsifying waxes. According to other embodiments, the composition comprises a combination of surfactants or emulsifying waxes. According to one embodiment, the emulsifier, selected from surfactants or emulsifying waxes include but not limited to: lecithin, glyceryl monostearate, glyceryl monooleate, cetyl alcohol, stearyl alcohol, cetostearyl alcohol, and the like, or combinations thereof. According to some embodiments, the composition comprises a combination of cetearyl alcohol and sodium lauryl sulfate. According to some embodiments, the ration of cetearyl alcohol to sodium lauryl sulfate is from 1:25 to 1:5, e.g. 1:20 to 1:9.

According to some embodiments, the composition may include from about 0.5 to about 15% by weight emulsifying waxes. According to some embodiments, the composition may include about 1-10% by weight emulsifying waxes. According to some embodiments, the composition may include about 2-7.5% by weight emulsifying waxes. According to some embodiments, the composition may include about 5% by weight of emulsifying waxes.

According to some embodiments, the composition may include from about 0.5 to about 15% by weight surfactants. According to some embodiments, the composition may include about 1-10% by weight surfactants. According to some embodiments, the composition may include about 2-7.5% by weight surfactants. According to some embodiments, the composition may include about 5% by weight of surfactants.

According to some embodiments, the composition may include from about 0.5 to about 15% by weight of a combination of surfactants and emulsifying waxes. According to some embodiments, the composition may include about 1-10% by weight of the combination of surfactants and emulsifying waxes. According to some embodiments, the composition may include about 2-7.5% by weight of the combination of surfactants and emulsifying waxes.

According to some embodiments, the composition may include about 5% by weight of of the combination of surfactants and emulsifying waxes. According to one embodiment, the combination comprises cetearyl alcohol and sodium lauryl sulfate.

According to some embodiments, the composition may include from about 0.5 to about 15% by weight of emulsifier. According to some embodiments, the composition may include about 1-10% by weight emulsifier. According to some embodiments, the composition may include about 2-7.5% by weight emulsifier. According to some embodiments, the composition may include about 5% by weight of emulsifier.

In some embodiments, the composition may further include Dittrichia viscosa oil extract. For example, the oil may be olive oil or any other suitable vegetable or dermatologically acceptable oil. According to some embodiments, the composition may include about 0.5-15% by weight of Dittrichia viscosa. According to some embodiments, the composition may include about 1-10% by weight Dittrichia viscosa. According to some embodiments, the composition may include about 2-7.5% by weight Dittrichia viscosa. According to some embodiments, the composition may include about 5% by weight of Dittrichia viscosa.

In some embodiments, the composition may further include Lavender oil extract. For example, the oil may any vegetable/dermatologically acceptable oil, such as, olive oil. According to some embodiments, the composition may include about 0.5-15% by weight of Lavender oil. According to some embodiments, the composition may include about 1-10% by weight Lavender oil. According to some embodiments, the composition may include about 2-7.5% by weight Lavender oil. According to some embodiments, the composition may include about 5% by weight of Lavender oil.

In some embodiments, the composition may further include Frankincense powder (resin powder). According to some embodiments, the composition may include about 0.5-15% by weight of Frankincense powder. According to some embodiments, the composition may include about 1-10% by weight Frankincense powder. According to some embodiments, the composition may include about 2-7.5% by weight Frankincense powder. According to some embodiments, the composition may include about 5% by weight of Frankincense powder.

In some embodiments, the composition may further include essential oils. In some embodiments, the essential oils may be a combination of one or more essential oils. In some embodiments, the essential oils may include Lavender, Clary sage, Myrrh and/or Frankincense. According to some embodiments, the composition may include about 0.5-10% by weight essential oils. According to some embodiments, the composition may include about 1-7.5% by weight essential oils. According to some embodiments, the composition may include about 2-5% by weight essential oils. According to some embodiments, the composition may include about 2% by weight of essential oil(s).

In some embodiments, the composition may further include additional whole plant extracts. In some embodiments, whole plant extracts may be obtained from one plant or from a combination of plants. In some embodiments, the whole plant extracts may include Ruta chalepensis, Dittrichia viscosa, and/or Moringa oleifera. According to some embodiments, the composition may include about 0.5-10% by weight of the whole plant extracts. According to some embodiments, the composition may include about 1-7.5% by weight whole plant extracts. According to some embodiments, the composition may include about 2-5% by weight whole plant extracts. According to some embodiments, the composition may include about 2% by weight of whole plant extracts.

According to some embodiments, the composition further comprises acacia gum. According to some embodiments, there is provided a topical skin care composition comprising therapeutically effective amounts of one or more of:
a) Cannabis, in the form of CBD oil;
b) Aloe vera gel;
c) Neem oil;
d) Optionally, at least one organosulfur compound selected from the group consisting of dimethyl sulfoxide (DMSO) and methylsulfonylmethane (MSM)
e) Optionally, Dittrichia viscosa in oil and Lavender in oil;
f) Optionally, Frankincense powder;
g) Optionally, at least one surfactant or emulsifying wax;
h) Optionally, at least one essential oil selected from the group consisting of Lavender, Clary sage, and Myrrh oils; and
i) Optionally whole plant extract, of one or more of Ruta chalepensis, Dittrichia viscosa, and Moringa oleifera;
   Each possibility is a separate embodiment.

In some embodiments there is provided a topical skin care composition:
a) 1-30% by weight CBD oil or at least one cannabinoid;
b) 7.5-50% by weight Aloe vera (gel or juice);
c) 0.5-15% by weight Neem oil;
d) Optionally, 15-55% by weight of at least one organosulfur compound selected from the group consisting of DMSO and MSM;
e) Optionally, 0.5-15% oil extracts of each of Dittrichia viscosa and Lavender;
f) Optionally, 0.5-15% Frankincense powder;
g) Optionally, 0.5-15% by weight at least one surfactant;
h) Optionally, 0.5-10% by weight of at least one essential oil selected from the group consisting of Lavender , Clary sage, and Myrrh oils; and
i) Optionally, 0.5-10% by weight of whole plant material extracts form one or more plants selected from the group consisting of Ruta chalepensis, Dittrichia viscosa, and Moringa oleifera.

Each possibility is a separate embodiment.

According to yet another embodiment, the composition comprises from 1 to 30% by weight CBD oil, from about 7.5 to about 50% by weight Aloe vera (gel or juice), from about 0.5 to about 15% by weight Neem oil, from about 15 to about 55% by weight of at least one organosulfur compound, e.g. DMSO or MSM, from about 0.5 to about 15% oil extract of Dittrichia viscosa, from about 0.5 to about 15% oil extract of Lavender, from 0.5 to 15 wt % of Frankincense powder, and optionally, (a) from 0.5 to about 15% by weight at least one emulsifier, (b) from about 0.5 to about 10% by weight of at least one essential oil, e.g. essential oil from Lavender , Clary sage, Myrrh oils, and any combination thereof, and/or (c) from about 0.5 to about 10% by weight of whole plant material extracts from one or more plants selected from the group consisting of Ruta chalepensis, Dittrichia viscosa, and Moringa oleifera.

According to some embodiments, the CBD oil comprises from about 10 to about 40 wt % CBD.

According to some embodiments, the composition comprises from 2.5 to 7.5 wt % emulsifiers such as emulsifying wax and surfactant(s), from 5 to 15 wt % CBD oil which was extracted e.g. from high CBD Cannabis strain, from 2.5 to 7.5 wt % Neem oil, from 2.5 to 7.5 wt % Frankincense powder (Boswellia serrata), from 2.5 to 7.5 wt % Dittrichia viscosa extract in oil, from 2.5 to 7.5 wt % of Lavender extract in oil, from 1 to 5 wt % of Essential oils from Lavender, Clary sage and Myrrh, from 1 to 5 wt % of whole plant material extract selected from from Ruta chalepensis extract, Dittrichia viscosa extract, Moringa oleifera extract, and combination thereof, from 30 to 50 wt % DMSO and from 15 to 25 wt % Aloe vera gel, wherein the content of all components sum in 100%. According to some embodiments, the CBD oil comprises from 40 to 80 wt %, from 50 to 80 wt % or from 55 to 75wt % of CBD. According to one embodiment, the CBD oil comprises from 55 to 80 wt % of CBD. According to another embodiment, the composition comprises from 2 to 22 wt % or from 4 to 15 wt % of CBD.

According to one embodiment, the composition comprises 5% w/w of a combination of emulsifying wax and surfactant, 10% w/w CBD oil which was extracted from high CBD Cannabis strain, 5% w/w Neem oil, 5% w/w Frankincense powder (Boswellia serrata), 5% w/w Dittrichia viscosa extract in oil, 5% w/w Lavender extract in oil, 2% w/w Essential oils from Lavender, Clary sage and Myrrh, 3% w/w whole plant material extract (Ruta chalepensis extract, Dittrichia viscosa extract, Moringa oleifera extract), 40% w/w DMSO and 20% w/w Aloe vera gel. According to some embodiments, the CBD oil comprises from 40 to 80 wt %, from 50 to 80wt % or from 55 to 75wt % of CBD. According to one embodiment, the CBD oil comprises from 55 to 80 wt % of CBD. According to another embodiment, the composition comprises from 2 to 22 wt % or from 4 to 15 wt % of CBD.

According to some embodiments, there is provided a composition wherein the composition comprises or consist of: about 5-25% by weight of CBD oil, about 10-30% by weight of Aloe vera extract, and about 2-10% by weight of Neem Oil and optionally one or more of: 20-55% by weight of at least one organosulfur compound selected from the group consisting of dimethyl sulfoxide (DMSO) and methyl sulfonylmethane (MSM); about 2-10% by weight of emulsifiers; about 2-10% by weight of Dittrichia viscosa in oil; about 2-10% by weight of Lavender in oil; about 2-10% by weight of Frankincense powder; about 0.5-5% by weight of essential oils, selected from the group consisting of: Lavender, Clary sage and Myrrh; about 0.5-5% by weight of whole plant extracts, said plants are selected from the group consisting of: Ruta chalepensis, Dittrichia viscosa, and Moringa oleifera. Each possibility is a separate embodiment. According to some embodiments, the CBD oil comprises from 40 to 80 wt %, from 50 to 80wt % or from 55 to 75wt % of CBD. According to one embodiment, the CBD oil comprises from 55 to 80 wt % of CBD. According to one embodiment, the at least one organosulfur compound is DMSO. According to another embodiment, the composition comprises from 2 to 22 wt % or from 4 to 15 wt % of CBD.

According to some embodiments, there is provided a composition comprising or consisting of: about 10% by weight of CBD oil, about 20% by weight of Aloe vera extract, and about 5% by weight of Neem Oil; about 40% by weight of at least one organosulfur compound selected from the group consisting of dimethyl sulfoxide (DMSO) and methylsulfonylmethane (MSM); about 5% by weight of emulsifying waxes and surfactants;

about 5% by weight of Dittrichia viscosa in oil; about 5% by weight of Lavender in oil; about 5% by weight of Frankincense powder; about 2% by weight of essential oils, selected from the group consisting of: Lavender, Clary sage and Myrrh; about 3% by weight of whole plant extracts, said plants are selected from the group consisting of: Ruta chalepensis, Dittrichia viscosa, and Moringa oleifera. Each possibility is a separate embodiment. According to some embodiments, the CBD oil comprises from 40 to 80 wt %, from 50 to 80wt % or from 55 to 75wt % of CBD. According to one embodiment, the CBD oil comprises from 55 to 80 wt % of CBD. According to another embodiment, the composition comprises from 2 to 22 wt % or from 4 to 15 wt % of CBD.

According to any one of the above embodiment, the composition may further comprise water or an aqueous phase. According to such embodiment, the composition further comprises a surfactant.

According to any one of the above embodiments, the composition of the present invention is a pharmaceutical composition further comprising a pharmaceutically acceptable carrier or excipient. According to other embodiment, the composition of the present invention is a cosmetic composition. According to one embodiments, the composition is a dermatological composition, e.g. a pharmaceutical dermatological composition.

According to another aspect, the composition of the present invention as described in any one of the above aspects and embodiments is for use in treatment of a dermatological condition in a subject in need thereof. Thus according to one embodiment, the composition, e.g. the pharmaceutical composition, comprising from about 1 to about 30 wt % of CBD oil, from about 5 to about 50 wt % of Aloe vera extract, and from about 0.5 to about 15% by weight of Neem oil, is for use in treatment of a dermatological condition. According to some embodiments, the composition further comprises a dermatologically or a pharmaceutically acceptable carrier. According to some embodiments, the dermatological condition is selected from atopic dermatitis, skin cancer lesions, psoriasis, herpes zoster, benign hyperproliferative disorders, typically superficial wounds, shallow ulcers, or combinations thereof. According to one embodiment, the dermatological condition is from atopic dermatitis. According to another embodiment, the dermatological condition is lesion. According to a further embodiment, the dermatological condition is herpes zoster. According to some embodiments, the dermatological condition is psoriasis. According to yet another embodiment, the dermatological condition is wound, ulcers or any other skin afflictions.

According to some embodiments, the composition of the present invention comprising from about 1 to about 30 wt % of CBD oil, from about 5 to about 50 wt % of Aloe vera extract, from about 0.5 to about 15% by weight of Neem oil, and from 20 to about 55% by weight of at least one organosulfur compound, is for use in treatment of a dermatological condition. According to another embodiment, the composition of the present invention comprising from about 5 to about 25% by weight of CBD oil, from about 10 to about 30% by weight of Aloe vera extract, from about 2 to about 10% by weight of Neem oil, and from about 20 to about 55% by weight of at least one organosulfur compound, is for use in treatment of a dermatological condition. According to some embodiments, the composition further comprises a dermatologically or a pharmaceutically acceptable carrier.

According to some embodiments, there is provided a composition for use in treatment of a dermatological condition in a subject in need thereof, wherein the composition include about 5-25% by weight of CBD oil, about 10-30% by weight of Aloe vera extract, and about 2-10% by weight of Neem oil and optionally one or more of: 20-55% by weight of at least one organosulfur compound selected from the group consisting of dimethyl sulfoxide (DMSO) and methylsulfonylmethane (MSM); about 2-10% by weight of emulsifiers; about 2-10% by weight of Dittrichia viscosa in oil; about 2-10% by weight of Lavender in oil; about 2-10% by weight of Frankincense powder; about 0.5-5% by weight of essential oils, selected from the group consisting of: Lavender, Clary sage and Myrrh; about 0.5-5% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition comprises or consist of: about 5-25% by weight of CBD oil, about 10-30% by weight of Aloe vera extract, and about 2-10% by weight of Neem oil and optionally one or more of: 20-55% by weight of at least one organosulfur compound selected from the group consisting of dimethyl sulfoxide (DMSO) and methylsulfonylmethane (MSM); about 2-10% by weight of emulsifying waxes and surfactants; about 2-10% by weight of Dittrichia viscosa in oil; about 2-10% by weight of Lavender in oil; about 2-10% by weight of Frankincense powder; about 0.5-5% by weight of essential oils, selected from the group consisting of: Lavender, Clary sage and Myrrh; about 0.5-5% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition includes about 5-25% by weight of CBD oil, about 10-30% by weight of Aloe vera extract, and about 2-10% by weight of Neem oil; about 20-55% by weight of at least one organosulfur compound selected from the group consisting of dimethyl sulfoxide (DMSO) and methylsulfonylmethane (MSM); about 2-10% by weight of a combination of emulsifying waxes and surfactant(s); about 2-10% by weight of Dittrichia viscosa in oil; about 2-10% by weight of Lavender in oil; about 2-10% by weight of Frankincense powder; about 0.5-5% by weight of essential oils, selected from the group consisting of: Lavender, Clary sage and Myrrh; about 0.5-5% by weight of whole plant extracts, said plants are selected from the group consisting of: Ruta chalepensis, Dittrichia viscosa, and Moringa oleifera. Each possibility is a separate embodiment.

According to some embodiments, there is provided a composition for use in treatment of a dermatological condition in a subject in need thereof, wherein the composition comprising or consisting of: about 5-25% by weight of CBD oil, about 10-30% by weight of Aloe vera extract, and about 2-10% by weight of Neem oil; about 20-55% by weight of at least one organosulfur compound selected from the group consisting of dimethyl sulfoxide (DMSO) and methylsulfonylmethane (MSM); about 2-10% by weight of a combination of emulsifying wax such as cetearyl alcohol and surfactant such as sodium lauryl sulfate; about 2-10% by weight of Dittrichia viscosa in oil; about 2-10% by weight of Lavender in oil; about 2-10% by weight of Frankincense powder; about 0.5-5% by weight of essential oils, selected from the group consisting of: Lavender, Clary sage and Myrrh; about 0.5-5% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition include about 10% by weight of CBD oil, about 20% by weight of Aloe vera extract, and about 5% by weight of Neem oil and optionally one or more of: about 40% by weight of at least one organosulfur compound selected from the group consisting of dimethyl sulfoxide (DMSO) and methylsulfonylmethane (MSM); about 5% by weight of a combination of emulsifying wax such as cetearyl alcohol and surfactant such as sodium lauryl sulfate; about 5% by weight of Dittrichia viscosa in oil; about 5% by weight of Lavender in oil; about 5% by weight of Frankincense powder; about 2% by weight of essential oils, selected from the group consisting of: Lavender, Clary sage and Myrrh; about 3% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition comprising or consisting of: about 10% by weight of CBD oil, about 20% by weight of Aloe vera extract, and about 5% by weight of Neem oil and optionally one or more of: about 40% by weight of at least one organosulfur compound selected from the group consisting of dimethyl sulfoxide (DMSO) and methylsulfonylmethane (MSM); about 5% by weight of emulsifying wax BP; about 5% by weight of Dittrichia viscosa in oil; about 5% by weight of Lavender in oil; about 5% by weight of Frankincense powder; about 2% by weight of essential oils, selected from the group consisting of: Lavender, Clary sage and Myrrh; about 3% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition include: about 10% by weight of CBD oil, about 20% by weight of Aloe vera extract, and about 5% by weight of Neem Oil; about 40% by weight of at least one organosulfur compound selected from the group consisting of dimethyl sulfoxide (DMSO) and methylsulfonylmethane (MSM); about 5% by weight of emulsifying wax BP; about 5% by weight of Dittrichia viscosa in oil; about 5% by weight of Lavender in oil; about 5% by weight of Frankincense powder; about 2% by weight of essential oils, selected from the group consisting of: Lavender, Clary sage and Myrrh; about 3% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to some embodiments, there is provided a composition for use in treatment of a dermatological condition in a subject in need thereof, wherein the composition comprising or consisting of: about 10% by weight of CBD oil, about 20% by weight of Aloe vera extract, and about 5% by weight of Neem Oil; about 40% by weight of at least one organosulfur compound selected from the group consisting of dimethyl sulfoxide (DMSO) and methylsulfonylmethane (MSM); about 5% by weight of emulsifying wax BP; about 5% by weight of Dittrichia viscosa in oil; about 5% by weight of Lavender in oil; about 5% by weight of Frankincense powder; about 2% by weight of essential oils, selected from the group consisting of: Lavender, Clary sage and Myrrh; about 3% by weight of whole plant extracts, said plants are selected from the group consisting of: Ruta chalepensis, Dittrichia viscosa, and Moringa oleifera. According to one embodiment, the at least one organosulfur compound is DMSO. Each possibility is a separate embodiment.

According to any one of the above embodiments the CBD oil comprises from 40 to 80 wt %, from 50 to 80 wt % or from 55 to 75 wt % of CBD. According to one embodiment, the CBD oil comprises from 55 to 80 wt % of CBD. . According to one embodiment, the CBD oil comprises from 10 to 40 wt % of CBD. According to another embodiment, the composition comprises from 2 to 22 wt % or from 4 to 15 wt % of CBD.

In some embodiments, the composition or the pharmaceutical composition of the invention is formulated for topical administration. The compositions of the present invention may be in any of the galenical forms normally used for topical administration, in particular in the form of solutions, lotions, gels, emulsions of liquid or semi-liquid consistency of the milk type, obtained by dispersion of a fatty phase in an aqueous phase (O/W) or vice versa (W/O), or suspensions or emulsions of soft, semi-liquid or solid consistency, of the cream or ointment type, or else microemulsions, microcapsules, microparticles or vesicular dispersions of ionic and/or nonionic type. According to some embodiments, formulations for topical administration include ointments, gels, creams, lotions, solutions, balm and skin patch. Exemplary suitable carriers include vegetable or mineral oils, white petrolatum, branched chain fats or oils, animal fats and waxes. Stabilizers, preservatives, humectants and antioxidants may also be included, as well as agents imparting color or fragrance, if desired.

According to some embodiments, the composition or the pharmaceutical composition of the present invention is topically administered.

In some embodiments, the compositions may conveniently be presented in unit dosage form, and may be prepared by any of the methods well-known in the art. The selected dosage form (for example, gel, ointment, cream, skin patch, etc.), may depend upon the desired therapeutic effect, type of lesion or ulcer and/or the duration of the treatment. According to some embodiments, the compositions of the present invention such as a pharmaceutical composition is administered 1, 2, 3, 4 or 6 times a day. According to one embodiment, the composition is administered for 1, 2, 3, 4, 5, 6, or 7 days, for 2, 3, 4, of 5 weeks, or for 1, 2, 3, 4, 5, 6, 7, 8, 9, 10, 11 or 12 months.

In some embodiments, the administration regime may be determined while taking into account the clinical condition of the subject, the affected skin site of administration, the scheduling of administration, the subject's age, gender, body weight and other factors relevant to clinicians of ordinary skill in the art.

In some embodiments, the compositions of the invention may be provided in a pack, a patch, or dispenser device, such as a kit. In addition, the kit may include one or more carrier(s) and/or excipients.

In some embodiments, the kit may include the composition of the invention and optionally one or more suitable excipients or carriers and instructions for use of the kit in the treatment of dermatological ulcers and lesions.

According to some embodiments, there is provided a method for treating a dermatological condition in a subject in need thereof, comprising administering to the subject the composition of the invention. Thus according to one embodiment, the present invention provides a method of treating a dermatological condition in a subject in need thereof, comprising administering a composition comprising from about 1 to about 30 wt % of CBD oil, from about 5 to about 50 wt % of Aloe vera extract, and from about 0.5 to about 15% by weight of Neem oil. According to another embodiment, the method comprises administering a composition of the present invention comprising from about 1 to about 30 wt % of CBD oil, from about 5 to about 50 wt % of Aloe vera extract, and from about 0.5 to about 15% by weight of Neem oil. According to some embodiments, the composition further comprises a dermatologically or a pharmaceutically acceptable carrier.

According to any one of the above embodiments, the composition comprises from 30 to 55 wt % DMSO of from 35 to 45 wt % DMSO.

According to another aspect, the present invention provides use of a composition comprising from about 1 to about 30 wt % of CBD oil, from about 5 to about 50 wt % of Aloe vera extract, from about 0.5 to about 15% by weight of Neem oil, for preparation of a medicament for treating a dermatological condition. According to one embodiment, the composition further comprises from 20 to about 55% by weight of at least one organosulfur compound such as DMSO.

According to some embodiments, the CBD oil comprises from 40 to 80 wt %, from 50 to 80wt % or from 55 to 75wt % of CBD. According to one embodiment, the CBD oil comprises from 55 to 80 wt % of CBD. According to another embodiment, the composition comprises from 2 to 22 wt % or from 4 to 15 wt % of CBD.

As used herein, the singular form “a,” “an,” and “the” include plural references unless the context clearly dictates otherwise. For example, the term “a compound” or “at least one compound” may include a plurality of compounds, including mixtures thereof.

The term “comprising” means that other steps and ingredients that do not affect the final result can be added. This term encompasses the terms “consisting of” and “consisting essentially of” and may be replaced by it.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

The following examples are presented in order to more fully illustrate some embodiments of the invention. They should, in no way be construed, however, as limiting the broad scope of the invention.

### EXAMPLES

### Example 1

### Plant Extraction Process

The following extraction process was employed to obtain the following oil plant extracts:
1. Plant material was ground in the presence of ethanol to form a mixture of ground plant material and ethanol.
2. The resulting mixture was incubated for several days, depending on the type of plant and ambient temperature.
3. After incubation, the mixture was filtered.
4. After filtration, the ethanol was evaporated to receive a concentrated extract.
5. For Cannabis extract only: the concentrated extract oil was heated in a vacuum oven in order to activate the CBD molecule.

The homogenous oil extract thus obtained was used for the preparation of the tested compositions.

### Example 2

### Compositions (Formulations) for Treatment of Skin Lesions

Formulations for topical application were prepared as described below, and tested on various skin lesions of subjects.

The first formulation prepared (herein identified as Formulation #1) comprised Coconut oil, alcohol extract of Curcuma longa and oil extract of high-THC Cannabis. The extraction process is described in Example 1 above.

Subjects treated with the formulation reported of inconsistent effects, and complained of staining of skin and clothes.

Formulation #2 comprised Jojoba oil, alcohol extract of Curcuma longa, oil extract of high-THC Cannabis and 20% DMSO. Patients reported better effects but still complained of staining of skin and clothes.

Formulations #3 and #4 comprised Jojoba oil, oil extract of either high-THC or high-CBD Cannabis, respectively, and 40% DMSO. Patients reported better effects for Formulation #4 (containing high CBD oil) compared to formulation #3 and the previous formulations tested. However, the patients complained of an itching sensation and irritation of the skin upon application of the formulations, which lasted for up to half an hour after application.

Formulation #5 comprised a 25% w/w oily phase, including an 6.25% w/w emulsifying wax, 10% w/w CBD oil (oil extract of high CBD Cannabis ), 3.75% w/w Frankincense powder, 2.5% Vitamin E and 30 drops of Essential oils; and a 75% w/w aqueous phase including 56.2% w/w DMSO and 18.75% w/w Aloe vera juice. Patients reported better effects for Formulation #5, but about 50% of the patients still complained on an itching sensation and stimulation of the skin.

Composition (Formulation) #6 included 5% w/w emulsifying wax BP (comprising cetearyl alcohol 75-100% and sodium lauryl sulfate 5-10%), 10% w/w CBD oil which was extracted from high CBD Cannabis strain, 5% w/w Neem oil, 5% w/w Frankincense powder (Boswellia serrata), 5% w/w Dittrichia viscosa extract in oil, 5% w/w Lavender extract in oil, 2% w/w Essential oils (Lavender, Clary sage, Myrrh), 3% w/w whole plant material extract (Ruta chalepensis extract, Dittrichia viscosa extract, Moringa oleifera extract), 40% w/w DMSO and 20% w/w Aloe vera gel. Patients reported better effects with no side effects.

In all above mentioned formulations, the concentration of CBD in CBD oil was between 55 to 80 wt %.

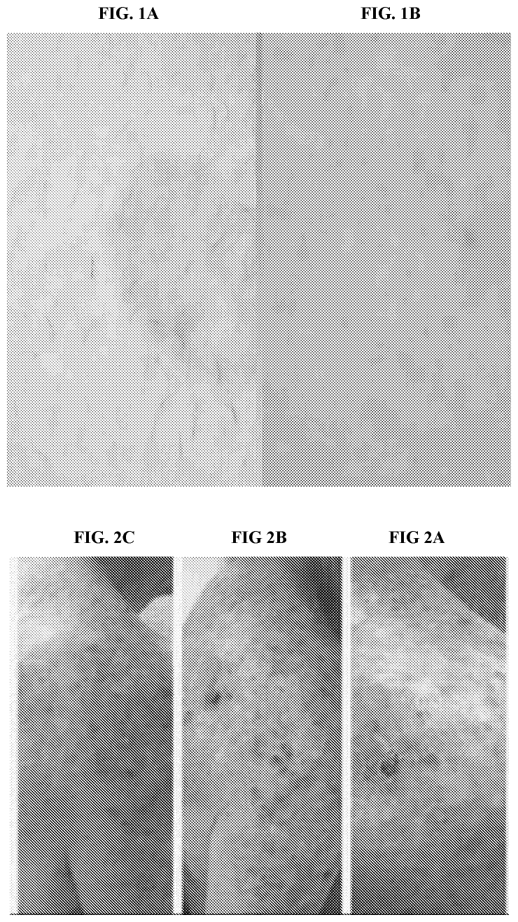

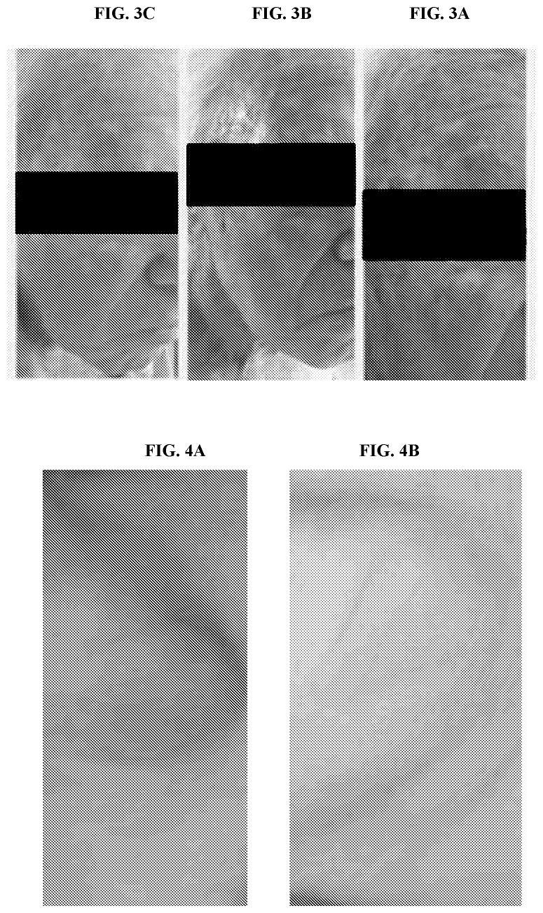

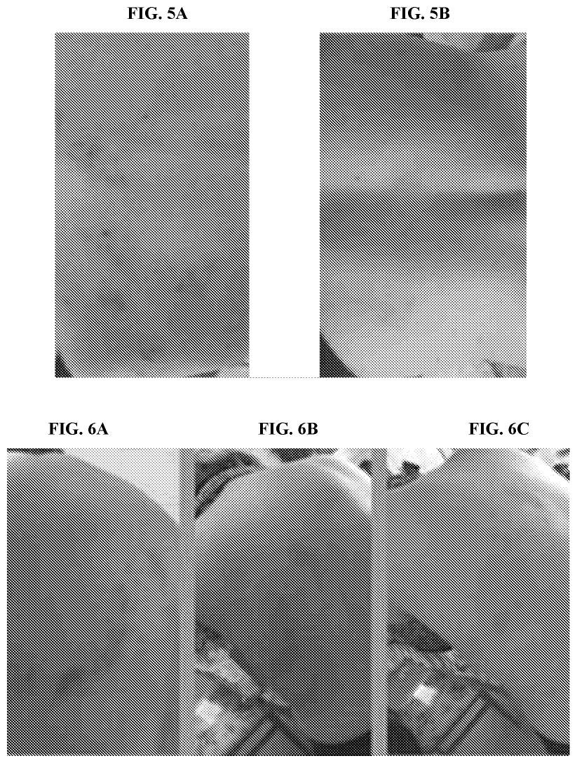

Formulation #6 was topically administered to subjects to treat various dermatological conditions, including: skin asthma (FIG. 1), skin lesions in cancer patients (FIG. 2 and FIG. 3), psoriasis (FIG. 4) and Shingles (Herpes Zoster, FIG. 5 and FIG. 6).

The composition (in the form of a cream) was applied locally to the skin lesions once a day. The administration is usually for 1-3 weeks, depending on the severity of the condition and the subject's response.

As can be seen in the results presented in FIGS. 1-6, clinical as well as cosmetic improvements were clearly demonstrated in all treated subjects. The improvements were visualized rapidly, within days from commencement of treatment, with no identified side effects.

As follows from FIG. 1, skin asthma lesions disappeared already on day 8. From FIG. 2 it can be seen that after 7 days of treatment skin lesion of a cancer patient reduced significantly and almost completely disappeared after 21 days of treatment.

FIG. 4 demonstrates that erythema caused by psoriasis was significantly reduced already after 2 days of treatments.

FIGS. 5 and 6 show that the symptoms of shingles were significantly improved after 5 days of treatment with Formulation #6.

All these results demonstrate that the composition of the present invention have a beneficial effect on a variety of dermatological conditions.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-25. (canceled)

26. A composition comprising from about 5 to about 25% by weight of CBD oil, from about 10 to about 30% by weight of Aloe vera extract, from about 2 to about 10% by weight of Neem oil, and from about 20 to about 55% by weight of at least one organosulfur compound.

27. The composition of claim 26, further comprising a dermatologically acceptable carrier.

28. The composition of claim 26, comprising from about 8 to about 12% or about 10% by weight of CBD oil.

29. The composition of claim 26, wherein the CBD oil comprises from about 40 to about 85% by weight or about 55 to about 80% by weight of CBD.

30. The composition of claim 26, wherein the CBD oil is obtained from plant extract, or is synthetically prepared.

31. The composition of claim 26, comprising from about 15 to about 25% by weight of Aloe vera extract.

32. The composition of claim 26, comprising from about 2 to about 8% by weight of Neem oil.

33. The composition of claim 26, wherein the at least one organosulfur compound constitutes from about 35 to about 45% by weight of the composition.

34. The composition of claim 26, wherein the at least one organosulfur compound is selected from the group consisting of dimethyl sulfoxide (DMSO) and methylsulfonylmethane (MSM).

35. The composition of claim 26, comprising from about 8 to about 15% by weight of CBD oil, from about 15 to about 25% by weight of Aloe vera gel, from about 2 to about 8% by weight of Neem oil, and from about 35 to about 45% of the at least one organosulfur compound selected from the group consisting of DMSO and MSM.

36. The composition of claim 35, wherein the CBD oil comprises from about 55 to about 80% by weight of CBD.

37. The composition of claim 26, further comprising from about 2 to about 10% by weight of an emulsifying wax, surfactant(s), or a combination thereof.

38. The composition of claim 26, further comprising from about 2 to about 10% by weight of oil extract(s) of Dittrichia viscosa and/or Lavender.

39. The composition of claim 26, further comprising from about 2 to about 10% by weight of Frankincense powder.

40. The composition of claim 26, further comprising from about 0.5 to about 5% by weight of at least one essential oil selected from the group consisting of Lavender, Clary sage, and Myrrh oil.

41. The composition of claim 26, further comprising from about 0.5 for about 5% by weight of whole plant extracts selected from the group consisting of Ruta chalepensis, Dittrichia viscosa, and Moringa oleifera.

42. The composition of claim 26, wherein the composition is characterized by at least one of (i) the composition is a pharmaceutical composition comprising one or more pharmaceutically acceptable excipients; (ii) the composition is formulated for topical administration; and (iii) the composition is in the form of an ointment, gel, skin patch, lotion, or cream.

43. A method for treating a dermatological condition in a subject in need thereof, comprising topically administering to the subject the composition of claim 26.

44. The method of claim 43, wherein the dermatological condition is selected from atopic dermatitis, skin cancer lesions, psoriasis, herpes zoster, benign hyperproliferative disorders, typically superficial wounds, shallow ulcers, and combinations thereof.
